(19) United States
(12) Patent Application Publication
(10) Pub. No.: US 2019/0320968 A1
(43) Pub. Date: Oct. 24, 2019

(54) Use of HP Xenon-129 MRI to Measure Xenon Signal Changes in the Brain Tissue Over a Period to Quantitatively Evaluate the Condition of CBF in an Individual

(71) Applicant: LAKEHEAD UNIVERSITY, Thunder Bay (CA)

(72) Inventors: Mitchell Albert, Thunder Bay (CA); Francis Hane, Thunder Bay (CA); Tao Li, Thunder Bay (CA)

(21) Appl. No.: 16/388,218

(22) Filed: Apr. 18, 2019

Related U.S. Application Data
(60) Provisional application No. 62/659,919, filed on Apr. 19, 2018.

(51) Int. Cl. A61B 5/00; A61B 5/026; A61B 5/055; G01R 33/56; G01R 33/563

(52) U.S. Cl. CPC A61B 5/4088; A61B 5/0263; A61B 5/055; A61B 5/0042; A61B 5/4082; A61B 2503/08; G01R 33/5601; G01R 33/56308; G01R 33/5608; A61B 2503/40; A61B 5/7275

(57) ABSTRACT

Described herein is the use HP xenon-129 MRI to measure xenon signal changes in the brain tissue over a period to quantitatively evaluate the condition of cerebral blood flow in an individual.

### PRIOR APPLICATION INFORMATION

The instant application claims the benefit of U.S. Provisional Patent Application Ser. No. 62/659,919, filed Apr. 19, 2018 and entitled “USE OF HP XENON-129 MRI TO MEASURE XENON SIGNAL CHANGES IN THE BRAIN TISSUE OVER A PERIOD TO QUANTITATIVELY EVALUATE THE CONDITION OF CBF IN AN INDIVIDUAL”, the entire contents of which are incorporated herein by reference for all purposes.

### BACKGROUND OF THE INVENTION

Current clinical diagnosis of AD relies on cognitive assessment at symptomatic stages. Such indirect and subjective method has a higher rate of false diagnosis. Alternatively, PET/SPECT are used as confirmatory and more definitive diagnosis modalities to detect disease-linked biomarkers such as amyloid plague, although their repeatability is greatly limited due to the radio-active tracers involved in the scans.

### SUMMARY OF THE INVENTION

According to one aspect of the invention, there is provided a method for measuring cerebral blood flow of an individual comprising:

ventilating the individual with a gas comprising hyperpolarized xenon gas;

generating a magnetic resonance spectra and/or image of brain tissue of the individual at an initial time point; then

generating at least one magnetic resonance spectra and/or image of the brain tissue at a later time point;

comparing a hyperpolarized xenon level in the magnetic resonance spectra and/or image of the brain tissue at the initial time point to a hyperpolarized xenon level in the magnetic resonance image of the brain tissue at the later time point, thereby determining cerebral blood flow of the individual.

According to another aspect of the invention, there is provided a method for diagnosing an individual who is at risk of developing a cerebral blood flow-related disorder for a cerebral blood flow-related disorder comprising:

ventilating the individual with a gas comprising hyperpolarized xenon gas;

generating a magnetic resonance spectra and/or image of brain tissue of the individual at an initial time point; then

generating at least one magnetic resonance spectra and/or image of the brain tissue at a later time point;

comparing a hyperpolarized xenon level in the magnetic resonance spectra and/or image of the brain tissue at the initial time point to a hyperpolarized xenon level in the magnetic resonance spectra and/or image of the brain tissue at the later time point, thereby determining cerebral blood flow of the individual; and comparing the cerebral blood flow rate of the individual to a cerebral blood flow rate of a healthy individual of similar age control, wherein if the cerebral blood flow rate of the individual is statistically lower than the cerebral blood flow rate of the healthy individual of similar age control, the individual is diagnosed with a cerebral blood flow-related disorder.

According to a further aspect of the invention, there is provided a method for determining if a compound of interest alters cerebral blood flow comprising:

ventilating a non-human test animal with a gas comprising hyperpolarized xenon gas;

generating a control magnetic resonance spectra and/or image of brain tissue of the non-human test animal at an initial time point; then

generating at least one control magnetic resonance spectra and/or image of the brain tissue at a later time point;

comparing a hyperpolarized xenon level in the control magnetic resonance spectra and/or image of the brain tissue at the initial time point to a control hyperpolarized xenon level in the magnetic resonance spectra and/or image of the brain tissue at the later time point, thereby determining a control cerebral blood flow of the individual;

administering a compound of interest to the non-human test animal and ventilating the non-human test animal with a gas comprising hyperpolarized xenon gas;

generating a test magnetic resonance spectra and/or image of brain tissue of the non-human test animal at an initial time point; then

generating at least one test magnetic resonance spectra and/or image of the brain tissue at a later time point;

comparing a hyperpolarized xenon level in the test magnetic resonance spectra and/or image of the brain tissue at the initial time point to a hyperpolarized xenon level in the test magnetic resonance spectra and/or image of the brain tissue at the later time point, thereby determining a test cerebral blood flow of the non-human test animal;

comparing the control cerebral blood flow of the non-human test animal to the test cerebral blood flow of the non-human test animal, wherein if the control cerebral blood flow of the non-human test animal and the test cerebral blood flow of the non-human test animal are different, the compound of interest alters cerebral blood flow.

According to yet another aspect of the invention, there is provided a method for evaluating progression of a cerebral blood flow-related disorder in an individual diagnosed with a cerebral blood flow-related disorder comprising:

ventilating the individual with a gas comprising hyperpolarized xenon gas;

generating a magnetic resonance spectra and/or image of brain tissue of the individual at an initial time point; then

generating at least one magnetic resonance spectra and/or image of the brain tissue at a later time point;

comparing a hyperpolarized xenon level in the magnetic resonance spectra and/or image of the brain tissue at the initial time point to a hyperpolarized xenon level in the magnetic resonance spectra and/or image of the brain tissue at the later time point, thereby determining current cerebral blood flow of the individual; and

comparing the current cerebral blood flow rate of the individual to a previous cerebral blood flow rate of the individual taken previously, wherein if the current cerebral blood flow rate of the individual is statistically lower than the previous cerebral blood flow rate of the individual, the cerebral blood flow-related disorder is progressing.

According to a still further aspect of the invention, there is provided a method for evaluating treatment efficacy of a cerebral blood flow-related disorder in an individual undergoing treatment for the cerebral blood flow-related disorder comprising:

ventilating the individual undergoing treatment for the cerebral blood flow-related disorder with a gas comprising hyperpolarized xenon gas;

generating a magnetic resonance spectra and/or image of brain tissue of the individual at an initial time point; then

generating at least one magnetic resonance spectra and/or image of the brain tissue at a later time point;

comparing a hyperpolarized xenon level in the magnetic resonance spectra and/or image of the brain tissue at the initial time point to a hyperpolarized xenon level in the magnetic resonance spectra and/or image of the brain tissue at the later time point, thereby determining current cerebral blood flow of the individual; and

comparing the current cerebral blood flow rate of the individual to a previous cerebral blood flow rate of the individual taken previously, wherein if the current cerebral blood flow rate of the individual is not statistically lower than the previous cerebral blood flow rate of the individual, the cerebral blood flow-related disorder is effective.

### BRIEF DESCRIPTION OF THE DRAWINGS

FIG. 1—Xenon wash-out time course of 129Xe signal in grey matter, from both healthy and AD participants.

FIG. 2—Derived beta parameter from xenon washout curves showing a difference in CBF between the two groups.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Xenon, when inhaled into the lungs, can dissolve into the blood and be carried to and then dissolved into the brain tissues (white matter and gray matter) via cerebral blood flow (CBF) across the blood brain barrier (BBB). Although chemically inert, xenon is biologically active and has long been acting as a general anesthetic. The time it takes for xenon to cumulatively dissolve into (wash-in) and exit brain tissues (wash-out) is directly related to and largely dominated by the condition of CBF. For example, a healthy individual may have a CBF of approximately about 50 ml/min/100 g of tissue.

Hyperpolarized (HP) Xenon MRI is an MR imaging technique that utilizes specially treated (hyperpolarized) xenon-129 gases as an imaging contrast to generate MR spectra and/or images of localized xenon perfusion in certain parts of the human body.

Described herein is the use HP xenon-129 MRI to measure xenon signal changes in the brain tissue over a period to quantitatively evaluate the condition of CBF in an individual.

Also described herein are methods for screening for compounds that alter CBF.

Also described herein are methods for diagnosing diseases associated with altered CBF in an individual, such as for example, but by no means limited to Alzheimer's disease (AD), Parkinson's disease or Frontotemporal Dementia.

Also described herein is a method for determining efficacy of a treatment administered to an individual suffering from an altered cerebral blood flow associated disease, for example, but by no means limited to Alzheimer's disease (AD), Parkinson's disease or Frontotemporal Dementia.

As discussed herein, in some embodiments, the invention is a method for early detection and diagnosis of Alzheimer's disease (AD). Specifically, this method uses hyperpolarized 129Xe MRI or NMR to quantitatively evaluate cerebral blood flow (CBF) changes by measuring the speed of xenon washing out of the brain tissues. Impaired CBF is one of AD's characteristic and has long been used for AD diagnosis. Because CBF starts to change at an early predementia stage of the disease, it has an advantage for early detection, compared to other biomarker-targeting (e.g. beta amyloid) technique.

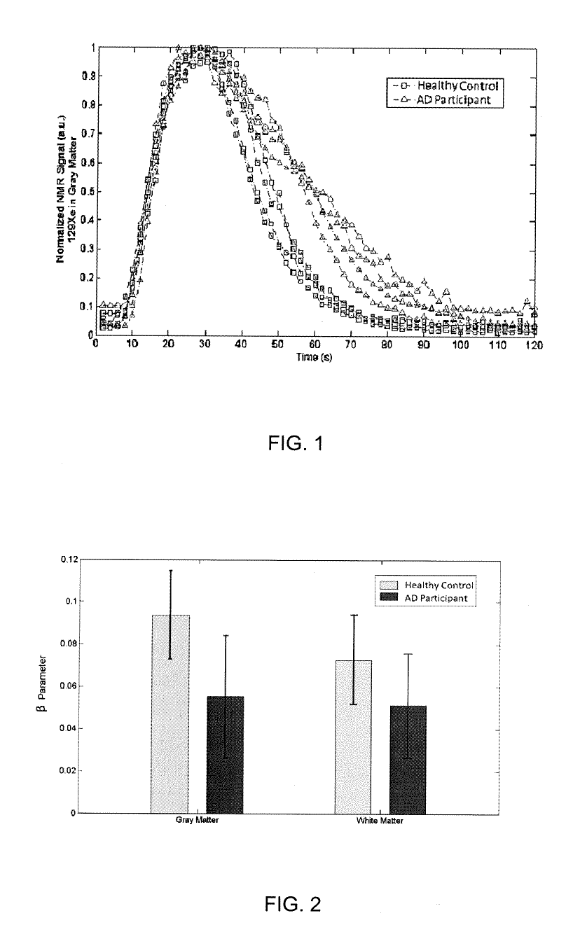

Representatively, FIG. 1 shows a time series xenon signals acquired from a group of healthy subjects and one with AD, with a visible difference in signal decreasing (xenon washout) time which is clearly visible at 50 seconds and becomes more pronounced between 60-80 seconds. FIG. 2 shows the cerebral blood flows from both groups, quantified and represented by the parameter derived from the washout curves, in comparison with each other, shown in bar graph form.

As will be appreciated by one of skill in the art, the “washout” of Xenon-129 can be measured by a variety of means known in the art, for example by MR spectroscopy or by MR imaging.

Furthermore, while a pre-determined amount of xenon-129 is administered to the individual or patient or subject or non-human test animal, the specific amount is not important as measurements are normalized based on an initial signal and it is the rate of change that is measured. That is, all that is required is that a sufficient amount of xenon-129 or an effective amount of xenon-129 is delivered to the brain tissue of the individual, patient, subject or non-human test animal for the method to be carried out.

In some embodiments, the rate of change or rate of xenon-129 washout is measured for at least 60 seconds.

In some embodiments, measurements are started prior to breath-hold by the patient, individual, subject or non-human test animal.

According to one aspect of the invention, there is provided a method for measuring cerebral blood flow of an individual comprising:

ventilating the individual with a gas comprising hyperpolarized xenon gas;

generating an initial magnetic resonance spectra and/or image of brain tissue of the individual at an initial time point; then

generating at least one later magnetic resonance spectra and/or image of the brain tissue at a later time point;

comparing a hyperpolarized xenon level in the initial magnetic resonance spectra and/or image of the brain tissue at the initial time point to a hyperpolarized xenon level in the at least one later magnetic resonance spectra and/or image of the brain tissue at the later time point, thereby determining cerebral blood flow of the individual.

In some embodiments of the invention, a plurality of magnetic resonance spectra and/or images are taken periodically following the initial time point so that a time course of change in hyperpolarized xenon is generated. In these embodiments, the time course is used to calculate the cerebral blood flow rate. As will be appreciated by one of skill in the art, the comparison is carried out as discussed above for each time point of the time course and an average rate may be generated from all of the comparisons or from all of the suitable comparisons.

As used herein, “initial” does not necessarily mean the “first” spectra and/or image generated in a series such as a time course but rather the “initial” may be a spectra and/or image generated “earlier” in the time course than the “later” spectra and/or image.

In some embodiments, the time course is at least 60 seconds, 60-90 seconds, about 60 seconds, about 90 seconds, about two minutes or about 60-120 seconds.

In some embodiments, the volume of brain tissue that is being imaged is measured or estimated for determining the cerebral blood flow rate.

As will be appreciated by one of skill in the art and as discussed herein, once the cerebral blood flow has been determined or measured or calculated, this can be used for diagnostic purposes.

For example, the calculated cerebral blood flow rate can be compared to a value typical for a healthy individual of a similar age. It is of note that unlike many other disorders, no factors other than age have been scientifically proven to be contributors to the development of diseases such as Alzheimer's disease. For example, a healthy individual may have a CBF of approximately about 50 ml/min/100 g of tissue and if the CBF of the individual is statistically significantly lower than this value or the corresponding age-related value, this indicates that the individual has or is at risk of developing or is developing a cerebral blood flow-related disorder such as for example but by no means limited to Alzheimer's disease (AD), Parkinson's disease or Frontotemporal Dementia.

Alternatively, the individual may be compared to a control, for example, a cerebral blood flow time course from a healthy individual of similar age. As will be appreciated by one of skill in the art, the control does not need to be repeated each time. In some embodiments, as discussed herein, the “control” may be CBF information and/or spectra and/or images generated as described herein of the individual previously. As discussed herein, by comparing current CBF to previous or historical CBF data for the individual, who may be a human patient, progression of CBF over time can be monitored, for example, for determining progression of the cerebral blood flow-related disorder or for determining the how successful a prescribed treatment is at treating the disease. As discussed herein, a prescribed treatment may include: prescription of medicaments and/or pharmaceutical compounds taken according to a dosage regimen; physical therapy; and/or an exercise plan, which may include physical exercise, memory or thinking exercises and the like.

In some embodiments, the hyperpolarized gas is hyperpolarized xenon-129 gas.

According to one aspect of the invention, there is provided a method for diagnosing an individual who is at risk of developing a cerebral blood flow-related disorder for a cerebral blood flow-related disorder comprising:

ventilating the individual with a gas comprising hyperpolarized xenon gas;

generating an initial magnetic resonance spectra and/or image of brain tissue of the individual at an initial time point; then

generating at least one later magnetic resonance spectra and/or image of the brain tissue at a later time point;

comparing a hyperpolarized xenon level in the initial magnetic resonance spectra and/or image of the brain tissue at the initial time point to a hyperpolarized xenon level in the at least one later magnetic resonance spectra and/or image of the brain tissue at the later time point, thereby determining cerebral blood flow of the individual; and

comparing the cerebral blood flow rate of the individual to a cerebral blood flow rate of a healthy individual of similar age control, wherein if the cerebral blood flow rate of the individual is statistically lower than the cerebral blood flow rate of the healthy individual of similar age control, the individual is diagnosed with a cerebral blood flow-related disorder.

In some embodiments of the invention, a plurality of magnetic resonance images are taken periodically following the initial time point so that a time course of change in hyperpolarized xenon is generated.

In some embodiments, the time course is at least 60 seconds, 60-90 seconds, about 60 seconds, about 90 seconds, about two minutes or about 60-120 seconds.

In some embodiments, the volume of brain tissue that is being imaged is measured or estimated for determining the cerebral blood flow rate.

The individual who is at risk of developing a cerebral blood flow-related disorder may be an individual who is of an advanced age, an individual who is showing symptoms of a cerebral blood flow-related disorder such as for example but by no means limited to Alzheimer's disease (AD), Parkinson's disease or Frontotemporal Dementia.

For example, the calculated cerebral blood flow rate can be compared to a value typical for a healthy individual of a similar age. It is of note that unlike many other disorders, no factors other than age have been scientifically proven to be contributors to the development of diseases such as Alzheimer's disease. For example, a healthy individual may have a CBF of approximately about 50 ml/min/100 g of tissue and if the CBF of the individual is statistically significantly lower than this value or the corresponding age-related value, this indicates that the individual has or is at risk of developing or is developing a cerebral blood flow-related disorder such as for example but by no means limited to Alzheimer's disease (AD), Parkinson's disease or Frontotemporal Dementia.

Alternatively, the individual may be compared to a control, for example, a cerebral blood flow time course from a healthy individual of similar age. Alternatively, the individual may be compared to their own CBF data generated previously, as discussed herein As will be appreciated by one of skill in the art, the control does not need to be repeated each time.

In some embodiments, the hyperpolarized gas is hyperpolarized xenon-129 gas.

In some embodiments of the invention, once the individual is diagnosed with a cerebral blood flow-related disorder, such as for example but by no means limited to Alzheimer's disease (AD), Parkinson's disease or Frontotemporal Dementia, appropriate treatments and/or interventions are carried out. In some embodiments, the individual is prescribed a treatment plan, as discussed herein, for example, a neurological disorder treatment plan. Specifically, the individual may meet with a neurological disorder specialist and be assigned a treatment plan, as discussed herein.

According to one aspect of the invention, there is provided a method for determining if a compound of interest alters cerebral blood flow comprising:

ventilating a non-human test animal with a gas comprising hyperpolarized xenon gas;

generating an initial control magnetic resonance spectra and/or image of brain tissue of the non-human test animal at a control initial time point; then

generating at least one later control magnetic resonance spectra and/or image of the brain tissue at a control later time point;

comparing a hyperpolarized xenon level in the initial control magnetic resonance spectra and/or of the brain tissue at the initial time point to a control hyperpolarized xenon level in the at least one later magnetic resonance spectra and/or of the brain tissue at the later time point, thereby determining a control cerebral blood flow of the individual;

administering a compound of interest to the non-human test animal and ventilating the non-human test animal with a gas comprising hyperpolarized xenon gas;

generating an initial test magnetic resonance spectra and/or of brain tissue of the non-human test animal at an initial time point; then

generating at least one later test magnetic resonance spectra and/or of the brain tissue at a later test time point;

comparing a hyperpolarized xenon level in the initial test magnetic resonance spectra and/or of the brain tissue at the initial time point to a hyperpolarized xenon level in the at least one later test magnetic resonance spectra and/or of the brain tissue at the later time point, thereby determining a test cerebral blood flow of the non-human test animal;

comparing the control cerebral blood flow of the non-human test animal to the test cerebral blood flow of the non-human test animal, wherein if the control cerebral blood flow of the non-human test animal and the test cerebral blood flow of the non-human test animal are different, the compound of interest alters cerebral blood flow.

In some embodiments of the invention, a plurality of magnetic resonance spectra and/or are taken periodically following the initial time point so that a time course of change in hyperpolarized xenon is generated.

In some embodiments, the time course is at least 60 seconds, 60-90 seconds, about 60 seconds, about 90 seconds, about two minutes or about 60-120 seconds.

In some embodiments, the volume of brain tissue that is being imaged is measured or estimated for determining the cerebral blood flow rate.

Alternatively, the individual may be compared to a control, for example, a cerebral blood flow time course from a healthy individual of similar age. As will be appreciated by one of skill in the art, the control does not need to be repeated each time.

In some embodiments, the hyperpolarized gas is hyperpolarized xenon-129 gas.

According to one aspect of the invention, there is provided a method for evaluating progression of a cerebral blood flow-related disorder in an individual diagnosed with a cerebral blood flow-related disorder comprising:

ventilating the individual with a gas comprising hyperpolarized xenon gas;

generating an initial magnetic resonance spectra and/or image of brain tissue of the individual at an initial time point; then

generating at least one later magnetic resonance spectra and/or image of the brain tissue at a later time point;

comparing a hyperpolarized xenon level in the initial magnetic resonance spectra and/or image of the brain tissue at the initial time point to a hyperpolarized xenon level in the later magnetic resonance spectra and/or image of the brain tissue at the later time point, thereby determining current cerebral blood flow of the individual; and

comparing the current cerebral blood flow rate of the individual to a historical cerebral blood flow rate of the individual taken previously, wherein if the current cerebral blood flow rate of the individual is statistically lower than the historical cerebral blood flow rate of the individual, the cerebral blood flow-related disorder is progressing.

In some embodiments of the invention, a plurality of magnetic resonance spectra and/or images are taken periodically following the initial time point so that a time course of change in hyperpolarized xenon is generated.

In some embodiments, the time course is at least 60 seconds, 60-90 seconds, about 60 seconds, about 90 seconds, about two minutes or about 60-120 seconds.

In some embodiments, the volume of brain tissue that is being imaged is measured or estimated for determining the cerebral blood flow rate.

The cerebral blood flow-related disorder may be for example but by no means limited to Alzheimer's disease (AD), Parkinson's disease or Frontotemporal Dementia.

In some embodiments, the hyperpolarized gas is hyperpolarized xenon-129 gas.

According to one aspect of the invention, there is provided a method for evaluating treatment efficacy of a cerebral blood flow-related disorder in an individual undergoing treatment for the cerebral blood flow-related disorder comprising:

ventilating the individual undergoing treatment for the cerebral blood flow-related disorder with a gas comprising hyperpolarized xenon gas;

generating an initial magnetic resonance spectra and/or image of brain tissue of the individual at an initial time point; then

generating at least one later magnetic resonance spectra and/or image of the brain tissue at a later time point;

comparing a hyperpolarized xenon level in the initial magnetic resonance spectra and/or image of the brain tissue at the initial time point to a hyperpolarized xenon level in the at least one later magnetic resonance spectra and/or image of the brain tissue at the later time point, thereby determining current cerebral blood flow of the individual; and

comparing the current cerebral blood flow rate of the individual to a historical cerebral blood flow rate of the individual taken previously, wherein if the current cerebral blood flow rate of the individual is not statistically lower than the historical cerebral blood flow rate of the individual, the cerebral blood flow-related disorder is effective.

In some embodiments of the invention, a plurality of magnetic resonance spectra and/or images are taken periodically following the initial time point so that a time course of change in hyperpolarized xenon is generated.

In some embodiments, the time course is at least 60 seconds, 60-90 seconds, about 60 seconds, about 90 seconds, about two minutes or about 60-120 seconds.

In some embodiments, the volume of brain tissue that is being imaged is measured or estimated for determining the cerebral blood flow rate.

The cerebral blood flow-related disorder may be for example but by no means limited to Alzheimer's disease (AD), Parkinson's disease or Frontotemporal Dementia.

In some embodiments, the hyperpolarized gas is hyperpolarized xenon-129 gas.

As will be apparent to one of skill in the art, one of the main advantages of this invention is its ability for early AD diagnosis with a non-radioactive technique that provides fast and reliable diagnostic results at early stages. Current clinical diagnosis of AD relies on cognitive assessment at symptomatic stages. Such indirect and subjective method has a higher rate of false diagnosis. Alternatively, PET/SPECT are used as confirmatory and more definitive diagnosis modalities to detect disease-linked biomarkers such as amyloid plague, although their repeatability is greatly limited due to the radio-active tracers involved in the scans.

The scope of the claims should not be limited by the preferred embodiments set forth in the examples but should be given the broadest interpretation consistent with the description as a whole.

1. A method for measuring cerebral blood flow of an individual comprising:
ventilating the individual with a gas comprising hyperpolarized xenon gas;
generating an initial magnetic resonance spectra and/or image of brain tissue of the individual at an initial time point; then
generating at least one later magnetic resonance spectra and/or image of the brain tissue at a later time point;
comparing a hyperpolarized xenon level in the initial magnetic resonance spectra and/or image of the brain tissue at the initial time point to a hyperpolarized xenon level in the at least one later magnetic resonance spectra and/or image of the brain tissue at the later time point, thereby determining cerebral blood flow of the individual.

2. The method according to claim 1 wherein a plurality of magnetic resonance spectra and/or images are taken periodically following the initial time point so that a time course of change in hyperpolarized xenon is generated.

3. The method according to claim 2 wherein the time course is at least 60 seconds, 60-90 seconds, about 60 seconds, about 90 seconds, about two minutes or about 60-120 seconds.

4. The method according to claim 1 wherein the volume of brain tissue that is being imaged is measured or estimated.

5. The method according to claim 1 wherein the cerebral blood flow is used for diagnostic purposes.

6. The method according to claim 1wherein the cerebral blood flow rate is compared to a value typical for a healthy individual of a similar age.

7. The method according to claim 1 wherein the cerebral blood flow-related disorder is selected from the group consisting of Alzheimer's disease (AD), Parkinson's disease and Frontotemporal Dementia.

8. The method according to claim 1 wherein the hyperpolarized gas is hyperpolarized xenon-129 gas.

9. The method according to claim 1 wherein the determined cerebral blood flow rate of the individual is compared to a historical cerebral blood flow rate of the individual taken previously.

10. A method for diagnosing an individual who is at risk of developing a cerebral blood flow-related disorder for a cerebral blood flow-related disorder comprising:
ventilating the individual with a gas comprising hyperpolarized xenon gas;
generating an initial magnetic resonance spectra and/or image of brain tissue of the individual at an initial time point; then
generating at least one later magnetic resonance spectra and/or image of the brain tissue at a later time point;
comparing a hyperpolarized xenon level in the initial magnetic resonance spectra and/or image of the brain tissue at the initial time point to a hyperpolarized xenon level in the at least one later magnetic resonance spectra and/or image of the brain tissue at the later time point, thereby determining cerebral blood flow of the individual; and
comparing the cerebral blood flow rate of the individual to a cerebral blood flow rate of a healthy individual of similar age control, wherein if the cerebral blood flow rate of the individual is statistically lower than the cerebral blood flow rate of the healthy individual of similar age control, the individual is diagnosed with a cerebral blood flow-related disorder.

11. The method according to claim 10 wherein a plurality of magnetic resonance spectra and/or images are taken periodically following the initial time point so that a time course of change in hyperpolarized xenon is generated.

12. The method according to claim 11 wherein the time course is at least 60 seconds, 60-90 seconds, about 60 seconds, about 90 seconds, about two minutes or about 60-120 seconds.

13. The method according to claim 10 wherein the volume of brain tissue that is being imaged is measured or estimated for determining the cerebral blood flow rate.

14. The method according to claim 10 wherein the individual who is at risk of developing a cerebral blood flow-related disorder is an individual who is of an advanced age, or an individual who is showing symptoms of a cerebral blood flow-related disorder.

15. The method according to claim 14 wherein the cerebral blood flow-related disorder is selected from the group consisting of Alzheimer's disease (AD), Parkinson's disease and Frontotemporal Dementia.

16. The method according to claim 10 wherein the hyperpolarized gas is hyperpolarized xenon-129 gas.

17. The method according to claim 10 wherein once the individual is diagnosed with a cerebral blood flow-related disorder, appropriate treatments and/or interventions are carried out.

18. A method for determining if a compound of interest alters cerebral blood flow comprising:
ventilating a non-human test animal with a gas comprising hyperpolarized xenon gas;
generating an initial control magnetic resonance spectra and/or image of brain tissue of the non-human test animal at an initial control time point; then
generating at least one later control magnetic resonance spectra and/or image of the brain tissue at a later control time point;
comparing a hyperpolarized xenon level in the initial control magnetic resonance spectra and/or image of the brain tissue at the initial control time point to a control hyperpolarized xenon level in the at least one later magnetic resonance spectra and/or image of the brain tissue at the later control time point, thereby determining a control cerebral blood flow of the individual;
administering a compound of interest to the non-human test animal and ventilating the non-human test animal with a gas comprising hyperpolarized xenon gas;
generating an initial test magnetic resonance spectra and/or image of brain tissue of the non-human test animal at an initial test time point; then
generating at least one later test magnetic resonance spectra and/or image of the brain tissue at a later test time point;
comparing a hyperpolarized xenon level in the initial test magnetic resonance spectra and/or image of the brain tissue at the initial test time point to a hyperpolarized xenon level in the at least one later test magnetic resonance spectra and/or image of the brain tissue at the later test time point, thereby determining a test cerebral blood flow of the non-human test animal;
comparing the control cerebral blood flow of the non-human test animal to the test cerebral blood flow of the non-human test animal, wherein if the control cerebral blood flow of the non-human test animal and the test cerebral blood flow of the non-human test animal are different, the compound of interest alters cerebral blood flow.

19. The method according to claim 18 wherein a plurality of magnetic spectra and/or resonance images are taken periodically following the initial time point so that a time course of change in hyperpolarized xenon is generated.

20. The method according to claim 19 wherein the time course is at least 60 seconds, 60-90 seconds, about 60 seconds, about 90 seconds, about two minutes or about 60-120 seconds.
